SEQUENCE LISTING (2 sequences)

SEQ ID NO: 1 - DNA, 5717 bp, Arabidopsis thaliana
  CDS (2484)..(4493)
tctagattatgtatttccaactttcattaacaatataatcgcatataaatgaaaaatcgtttccaggataatattttgatgaaatctcatattattgttcgtactcggattgatgttgaaggcttgaagcgcttcaaattatagaccagattatttaagtttttcttttgtttactccatatcaatttgatccattatactacctaagaaaatttaggtaacatagaattatttattgttatagtaaaaaaaaggaaaaccacaaaaataatctacttttacgtatatactattttcatgacataagtaattaagttgtacaacttttttttaatgaaaagagagagtaaatttatcatgttcatgtgtagttacctcgtgaataaccgacggttatatagacgcctaacatgaattgttcagttgaagacagttcaaaacatgtgtttcactctaaaatcctcaacaaaaaaaaagtgttaaaatttgtaaacctctttcaagcaaaaaaagaaaaagtgttagaatcccaagattctttcataatccggaatcttggctgaaaacgtataaaagagattgacgtagtaacaaggagtcttggtatgcttccatgctttttatccttttttgtcatggaaccatgatttggttaccatttattatgtaaccgaaattttcattgtaataatgaatatttaaatttttagcaaaaaaaaacaaaaaaaaacaaggagtcttgtcttcgttctcaaatttcagagctcttgcacttttcaagagttttactttgatgagtgagacatttgtctttttagtgtttattttctaaacttaaaatagtagcatcaacatcactcaattataattcttaagatgttgtagaaaaatattttatagatggaaagtaatcgatattaagacaaataagaaaccaaaccggactttgtgttcagaccgaatcaaatctgaattggagaaattatggtggaggcgaaagtcaacggaactaaagtataaaaccaaatgtcaaaaataaaacccaattttcatccttaaacgaacctgctgaaaccctaatttcgattaccaattccgatctaaaaagaagtcatggaagccattgattccgcaatcgatcctctcagagatttcgctaagagcagtgttcgtctcgtccagcgctgtcacaaacccgatcgcaagggtaacgccttttctcaaaaaaatctcatttccgatttttgatctgtagattagggttttctgaaattttgatatcatttgtaattgaattggttatcagaattcacgaaagtagctgtgcgtacggcgattggatttgtggtgatgggattcgttggattcttcgtgaagctcgttttcatcccaatcaacaacatcatcgttggatcttcttagtgtagtactttctttacgaggtaattgatctcgcattatatatctacattttggttatgttacttgacatatagtcattgattcaatagttctgttaattcctttaaagatcattttgactagaccacattcttggttcattcctcaataatttgtaatcatattggtggatatagaagtagattggttatagatcagatagtggaagactttaggatgaatttcagctagtttttttttttggcttattgtctcaaaagattagtgctttgctgtctccattgcttctgctatcgacacgcttctgtctccttgtatctttattatatctattcgtcccatgagttttgtttgttctgtattcgttcgctctggtgtcatggatggagtctctgttccatgtttctgtaatgcatgttgggttgtttcatgcaagaaatgctgagataaacactcatttgtgaaagtttctaaactctgaatcgcgctacaggcaatgctccgaggagtaggaggagaagaacgaaccaaacgacattatcagccctttgaggaagctcttagttttgttattgtttttgtagccaaattctccattcttattccattttcacttatctcttgttccttatagaccttataagttttttattcatgtatacaaattatattgtcatcaagaagtatctttaaaatctaaatctcaaatcaccaggactatgtttttgtccaattcgtggaaccaacttgcagcttgtatccattctcttaaccaataaaaaaagaaagaaagatcaatttgataaatttctcagccacaaattctacatttaggttttagcatatcgaaggctcaatcacaaatacaatagatagactagagattccagcgtcacgtgagttttatctataaataaaggaccaaaaatcaaatcccgagggcattttcgtaatccaacataaaacccttaaacttcaagtctcatttttaaacaaatcatgttcacaagtctcttcttcttctctgtttctctatctcttgctcatctttctcctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctaga

SEQ ID NO: 2 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPNGGTFNDVITEGDGRIKY

(19) United States
(12) Patent Application Publication
(10) Pub. No.: US 2006/0174366 A1
(43) Pub. Date: Aug. 3, 2006

(54) Use of ahas mutant genes as selection marker in potato transformation

(76) Inventors: Mariette Andersson, Lund (SE); Adelina Trifonova, Kavlinge (SE); Per Hofvander, Falsterbo (SE)

Correspondence Address: MORRISON & FOERSTER LLP, 1650 TYSONS BOULEVARD, SUITE 300, MCLEAN, VA, 22102, US

(21) Appl. No.: 10/519,947

(22) PCT Filed: Jul. 3, 2003
(86) PCT No.: PCT/EP03/07085

§ 371 Date: Dec. 29, 2004

(30) Foreign Application Priority Data
Jul. 9, 2002 (EP) 02015247.6

(51) Int. Cl. A01H 5/00; C12N 5/04; C12N 15/82

(52) U.S. Cl. 800/278; 435/468; 435/419; 800/317.2

(57) ABSTRACT

The present invention relates to the use of mutated AHAS genes conferring resistance to herbicides and resulting in an highly efficient selection system for the production of transgenic potato lines. The invention provides a key advantage by minimizing the escapes to almost zero of all shoots regenerated without interfering with the high number of shoots regenerated per explant.

The present invention relates to an improved selection system, sing a mutated acetohydroxy acid synthase (AHAS) gene, for production of transgenic potato lines. Genetic insertion of mutated HAS genes can give plants tolerance to a number of herbicide compounds. This invention yields a higher transformation efficiency and a reduced escape rate than previously described selection systems for patato plants.

The invention relates to a method for the generation of stably transformed fertile plants of the genus Solanum, which comprises the following steps:
(a) growing the plant to be transformed, and obtaining the suitable explant,
(b) transferring DNA sequences into plant cells,
(c) selecting transformed plant cells and
(d) regenerating transgenic fertile plants.

Moreover, the method comprises the possibility of transferring homologous or heterologous DNA sequences into the plant to be transformed or explants thereof, for example by Agrobacterium tumefaciens.

Moreover, the method comprises the possibility of using leaves as explants.

Acetohydroxyacid synthase (EC 4.1.3.18; AHAS; acetolactate sythase) is an enzyme catalysing, in two parallel pathways, the first step of the synthesis of the branched-chain aminoacids valin, leucin and isoleucin. In the valin and leucin biosynthesis, AHAS is catalysing the production of acetolactate by condensation of two pyruvate molecules. While in the isoleucin biosynthesis AHAS condensates one pyruvate molecule with one 2-oxobutyrat molecule to form acetohydroxybutyrat (Umbarger, 1975). Sequence comparison of the AHAS gene in higher plants shows high conservation in at least 10 regions. These regions are probably of great importance for the AHAS function. In tobacco two unlinked genes named SuRA and SuRB code for the AHAS enzyme catalytic subunit. This is not the case in Arabidopsis thaliana where only one gene is coding for the enzyme.

AHAS is the target enzyme of several classes of herbicides including sulphonylureas (Ray, 1984), imidazolinones (Shaner et al, 1984), triazolopyrimidines (Subrimanian et al, 1989) and pyrimidinyl oxybenzoat (Hawkes et al, 1989). The herbicides prevent branched amino acids to be synthesised by the plant and may lead to plant death. However, mutations in the AHAS genes can result in higher tolerance to these herbicides (U.S. Pat. No. 5,013,659) because of reduced affinity between enzyme and the herbicide.

In plants mutations conferring resistance to herbicides occur as a result of exposure to the compound repeatedly as it was reported for Arabidopsis thaliana. Once the mutated genes are isolated they can be used to genetically engineer plants for improved tolerance to the herbicides. For example mutations in the Arabidopsis thaliana AHAS gene have been produced and successfully confer resistance to imidazolinones as described in WO 00/26390, U.S. Pat. No. 5,767,366 and U.S. Pat. No. 6,225,105. Mutations in a corn AHAS gene confer imidazolinone resistance to monocot plants as described in EP 0 525 384.

The mutated AHAS genes can be used for production of herbicide resistant plants, yielding field resistance to a specific herbicide or can be used as a selection marker for genetic engineering of plants.

When genetic material is introduced into a population of plant cells, only a minor part of the cells are successfully transformed. For the production of novel genetically modified plants a selection system is transformed together with the trait genes providing the transformed cells with a selective growth advantage. This construct allows to select transformed from non-transformed cells by adding a compound favoring the regeneration of transformed shoots.

A number of selection systems have been developed; most of them are based on the use of herbicide or antibiotic resistance genes. For many plant species, several of the used selectable marker genes yield a low transformation efficiency and many non-transgenic escapes. Thus more efficient selection systems are required.

The use of antibiotic and herbicide resistance is called negative selection due to that the non-transformed cells are greatly retarded in growth or even killed. Selectable marker genes which can be used are for example the bialaphos resistance gene (bar) and the kanamycin or G418 resistance gene (NPTII). By growing the tissue on media containing for example kanamycin, in theory only the transformed cells will divide and take part in morphogenesis. The NPTII gene encodes a neomycin phosphotransferase, which reduces the inhibitory action of kanamycin, neomycin, G418 and paromomycin by a phosphorylation reaction.

In contrast to negative selection research has also been done on positive selection, based on giving the transformed cells a metabolic advantage while the non-transformed cells are starving with a concomitant slow reduction in viability. An example is the use of the mannose or xylose based selection system as described e.g. in U.S. Pat. No. 5,767,378.

Solanum tuberosum is one of the major target crops for genetic engineering. Main traits for potato are tuber quality, nutritional composition, starch quality, starch yield, insect and virus resistance. One trait with high market potential is the production of high amylopectin starch. The high amylopectin starch has an improved performance in the adhesive and paper industry compared to ordinary starch. In the paper production it will be used as a binder and for coating with printing quality better than latex used today.

Solanum tuberosum is a tetraploid plant with a high level of genetic heterozygosity. Conventional breeding of potato is therefore complicated because of segregation of important characteristics. Genetic engineering has during the last decade been an alternative to conventional breeding when it comes to improving potato varieties. A single trait or a combination of traits are more efficiently introduced by transformation than by using conventional breeding. Due to the fact that genetically modified potato plants can efficiently be vegetatively propagated the trait is fixed in the commercial line without the normal subsequent breeding.

Agrobacterium tumefaciens mediated transformation has successfully been achieved in potato since 1986. Almost exclusively the nptII gene has been used as a selection marker in potato.

The highest documented efficiency using nptII as selection marker in potato transformation is described by Libiakova, G. et al. in 2001. They have produced 120 leaf discs. Of those 81% produced one or more shoots. 90% of the shoots were confirmed transformants and showed resistance to kanamycin. This is equivalent to a regeneration efficiency of 81%, an escape rate of 10% and yields a transformation efficiency of 73%.

It is an object of the present invention to provide an efficient method for transformation of potato plants with a low escape rate and a high transformation efficiency.

Leaf segments are the explants of choice used in Agrobacterium mediated potato transformation. Transgenic plants are regenerated via direct or indirect organogenesis under selective conditions. Cytokinines as growth hormones are mainly involved in provoking cell division and adventitious bud formation in in vitro potato leaf segments. When Imazamox—(RS)-2-(4-isopropyl-4-methyl-5-oxo-2-imidazolin-2-yl)-5-methoxymethylnicotinic acid, an imidazolinone type herbicide—is applied in low concentrations it was surprisingly found that it possesses a cytokinine-like effect in tissue culture, increasing plant cell growth. At higher concentrations the substance is acting as an herbicide. The invention shows that Imazamox can efficiently be used as a selective agent in the recovery of transgenic potato plants and as a cytokinine-like substance it is supporting regeneration during transformation. This phenomenon classifies Imazamox as a unique selection agent that is combining both lethality for non-transgenic cells and improved growth for transgenic cells.

The present invention is describing a new selection system for efficient recovery of transgenic plants. The invention is also describing an innovative selection system that is acting as restriction for untransformed cells and support for transformed ones. Finally the invention is presenting an efficient system for recovery of transgenic potato plants and yields only a very small escape rate.

The present invention relates to the use of a mutated AHAS gene conferring imidazoline type herbicide resistance resulting in a highly efficient selection system for production of transgenic potato lines.

The invention provides a key advantage by minimizing the escapes to almost zero of all shoots regenerated without interfering with the high number of shoots regenerated per explant.

Another advantage is that the method does not comprise a gene conferring resistance to an antibiotic.

For example a mutated gene coding for a functional AHAS enzyme with tolerance to the imadazolinone type herbicides as described in Sathasiavan, K. et al, 1991 is used, see SEQ-ID No. 1. The mutated AHAS gene is used for insertion as a selection marker together with any other homologous or heterologous gene into the potato genome for production of transgenic potato plants. The AHAS enzyme naturally exists in higher plants and the use of a modified plant AHAS gene as selection marker conferring resistance to AHAS inhibiting herbicides does not result in the addition of a new biosynthetic function.

Furthermore
(i) the mutated AHAS gene can be of eukaryotic or prokaryotic origin;
(ii) the mutated AHAS gene can be regulated by any promoter functional in plants;
(iii) any plant transformation method for insertion of the gene constructs can be used;
(iiii) any mutations in the AHAS gene resulting in tolerance to an AHAS inhibiting herbicide can be used;
(iiiii) any potato variety can be transformed.

A mutated AHAS gene with mutation S653N as described in SEQ-ID No. 1 and U.S. Pat. No. 5,767,366 was cloned in a vector with the reporter gene beta-glucuronidase (GUS) (Jefferson, R. A. et al., 1987) and transformed to potato cells, see example 1 to 4. The GUS activity is tested in the transgenic lines produced and results in a transformation frequency of 93 to 100% of all transgenic shoots analysed. The transformation method produces a very high amount of shoots per explants, 3 to 5 independent transgenic shoots dependent on the variety used. With a calculation on transformation efficiency per explants the number can be as high as 500%. This is the highest transformation frequency of potato published. The mutated AHAS gene has also been used in combination with a gbss antisense gene for production of high amylopectin potato lines, see example 12. The AHAS selection system yielded in this case the same high transformation efficiency, which establishes the use of the AHAS gene as a selection marker also for commercially important traits.

The highest efficiency of nptII as a selection marker is 74% (see example 10) of totally analyzed shoots yielding a transformation efficiency of 370% at the maximum. Another selection system that can be used for potato transformation is the Mannose or xylose based selection system. Haldrup et al. (1998) have used the xylose isomerase gene in potato transformation, which yielded a transformation efficiency of 29% based on number of explants producing GUS positive shoots. High percentage of shoots positively transformed has been published for sugar beet and tobacco when using a very high mannose concentration, however the high concentration of the selective agent inhibit the number of shoots per explant distinctively (Joersbo et al., 1998).

The process furthermore comprises the possibility that compounds with herbicidal activity inhibiting the AHAS synthase can be employed for the selection of transgenic potato plants by using different combinations of growth regulators as for example auxins, cytokinines and/or auxin or cytokinines conjugates for the regeneration into intact transgenic plants.

The method is suitable for transforming the species Solanum tuberosum.

The invention also relates to transgenic fertile Solanum plants themselves which have been generated by the method described and to their transgenic seeds.

The method according to the invention allows for the first time to transform plant cells of the genus Solanum with a transformation efficiency of more than 93% to be regenerated into transgenic plants as shown in example 9.

The method for the generation of stably transformed Solanum plants is composed of the following steps: (a) growing the plant to be transformed and obtaining the suitable explant, (b) transferring DNA sequences into plant cells, (c) selecting transformed plant cells with compounds which inhibit in non transformed plant cells the AHAS synthase and (d) regenerating these transformed plant cells into fertile transgenic plants.

A variety of methods is currently available for the transformation. The most frequently employed method for transforming dicotyledonous plants is the Agrobacterium-tumefaciens-mediated gene transfer. This method exploits the natural ability of the soil bacterium to integrate genetic material into the plant genome. Other suitable methods are for example protoplast transformation by polyethylene-glycol-induced DNA uptake, electroporation, sonication or microinjection, the transformation of intact cells or tissue by micro- or macroinjection into tissue or embryos, tissue electroporation, incubation of dry embryos in DNA-containing solution, vacuum infiltration of seeds and the biolistic gene transfer.

The use of Agrobacterium tumfaciens for the transformation of plants using tissue culture explants has been described by Horsch et. al., Science 228(1985), 1229-1231; Fraley et al., Proc. Natl. Acad. Sci. USA 80(1983), 4803-4807 and Bevans et al., Nature 304(1983), 184-187. Many strains of Agrobacterium tumfaciens are capable of transferring genetic material into Solanum species such as for example the strains EHA 101, EHA105, LBA4404 and C58C1 with various disarmed Ti-plasmids.

The agrobacterial strain employed for the transformation of potatoes contains in addition to its disarmed Ti-plasmid a binary plasmid with the T-DNA to be transferred which contains a gene for selecting the transformed cells and the gene to be transferred. Both genes must be equipped with transcriptional and translational initiation and termination signals. The binary plasmid can be transferred into the agrobacterial strain for example by electroporation or other transformation methods, see Mozo & Hooykaas, Plant Mol. Biol. 16(1991), 917-918. Coculture of the plant explants with the agrobacterial strain takes two to three days.

Foreign genes can be expressed in a constitutive, inducible (by biotic and abiotic factors), tissue-specific or development-specific way. A relatively constitutive expression is achieved for example in plants by the cauliflower mosaic virus 35S promoter. This expression has been described by Shewmaker et al., Virology 140, 281-288 (1985) and Gardner et al., Plant Mol. Biol. 6, 221-228(1986).

Both the direct and the indirect gene transfer are. suitable transformation methods. The Agrobacterium-mediated transformation using a wide range of starting explants such as for example cotyledons, leaves, hypocotyls, shoots, roots, callus, mature and immature seeds or floral tissue can successfully be employed. The gene transfer can be effected both by simple cocultivation/incubation or wetting with the agrobacterial strain and by a supporting vacuum infiltration of the explants with the bacterial culture in question. The use of feeder cultures as aids may be advantageous in the process. Each agrobacterial strain which contains a Ti— or Ri-plasmid with the genetic information required for the transfer is suitable as vector for the transformation. Suitable agrobacterial strains are for example EHA101[pEHA101], EHA105[pEHA105], LBA4404[pAL4404], C58C1[pMP90] and C58C1[pGV2260]. Viral vectors also seem to be suitable for the transformation of Solanum. Other methods for transferring genetic material into Solanum are for example the polyethylene glycol (PEG)-mediated protoplast transformation, the electroporation, the sonication or microinjection and the transformation of intact cells or tissue by micro- or macroinjection into tissue or embryos, tissue electroporation, incubation of dry embryos in DNA-containing solution, the vacuum infiltration of seeds, see also Eds. Galun, E. and Breiman, A. In: Transgenic Plants, Imperial College Press, 1997. The use of the particle gun for bombarding a wide range of explants such as for example leaves and callus, is also possible.

All gene transfer vectors which contain the border sequences necessary for transferring the T-DNA (left and right border, LB and RB respectively), carry one or more selectable marker or reporter genes under the control of suitable promoters and terminators and/or contain further useful genes or target genes, also under the control of suitable transcriptional and translational regulatory units, can be employed for the Agrobacteria-mediated transformation. The transfer of DNA sequences into the Solanum plant to be transformed or explants thereof is preferably done by Agrobacterium-tumfaciens-mediated gene transfer.

The regeneration medium according to the invention furthermore comprises growth regulators such as auxins and/or auxin conjugates, and cytokinins and/or cytokinin conjugates. Suitable auxins are both natural and synthetic auxins. The natural auxin is indoleacetic acid (IAA), examples of synthetic auxins are 3-indolebutyric acid (IBA), 1-naphthylacetic acid (NAA) and the herbicide 2,4-dichlorophenoxyacetic acid (2,4-D). Other herbicides which act as auxins are also feasible as growth regulators. Auxin conjugates are compounds of auxin (IAA) with aspartic acid, glucose, myoinositol and others. In the case of the cytokinins, again, natural cytokinins such as, for example, zeatin and synthetic components, such as 6-benzylaminopurine (BAP) and 6-furfurylaminopurine (kinetin) may also be employed. Zeatin riboside is frequently employed as cytokinin conjugate. The auxins and the cytokinins can be employed in each case as individual components, but also as auxin or cytokinin mixtures. The concentration of the individual growth regulators is 0.05 to 10 mg/l, preferably 0.1 to 5 mg/l.

The transformation method according to the invention can be applied, inter alia, to Solanaceae species as Solanum tuberosum (potato), Lycopersicon esculentum (tomato) and pepper.

The mutant AHAS genes of the present invention confer resistance to imidazolinone herbicides. Types of herbicides to which resistance is conferred are described for example in U.S. Pat. Nos. 4,188,487; 4,201,565; 4,221,586; 4,297,128; 4,554,013; 4,608,079; 4,638,068; 4,747,301; 4,650,514; 4,698,092; 4,701,208; 4,709;036; 4,752;323; 4,772,311 and 4,798,619.

Mutant AHAS genes conferring resistance to AHAS inhibiting herbicides are described in U.S. Pat. No. 5,013,659, U.S. Pat. No. 5,141,870 and U.S. Pat. No. 5,378,824.

Other mutant AHAS genes of the present invention could also confer resistance to sulfonylurea herbicides. Types of mutants which confer sulfonylurea resistance are described for example in U.S. Pat. No. 5,853,973 and U.S. Pat. No. 5,928,937.

Mutant AHAS genes conferring resistance to imidazolinone type herbicides are described in WO 00/26390 and U.S. Pat. No. 5,767,366.

Furthermore Duggleby, R. G. and Pang, S. S. in Journal of Biochemistry and Molecular Biology 33(1), 1-36 (2000) describe mutations of the AHAS genes which could be used in the invention for conferring herbicide resistance to transgenic potato plants.

In WO 00/26390 additional genomic and cDNA sequences coding for an eukaryotic AHAS small subunit protein are disclosed. The DNA sequences and vectors are used to transform plants to produce transgenic plants which possess elevated levels of tolerance or resistance to herbicides such as imidazolinones.

It will be understood by those working in the field that the nucleic acid sequence depicted in SEQ-ID No. 1 is not the only sequence which can be used to confer imidazolinone-specific resistance. Also contemplated are those nucleic acid sequences which encode an identical protein but which, because of the degeneracy of the genetic code, possess a different nucleotide sequence. The invention also encompasses genes encoding AHAS sequences in which the above-mentioned mutation is present, but which also encode one or more silent amino acid changes in positions of the molecule not relevant for resistance to herbicides or to the catalytic function. Also contemplated are gene sequences from other imidazolinone resistant monocot or dicot plants which have a mutation in the corresponding region of the sequence.

For example, alterations in the gene sequence which results in the production of a chemically equivalent amino acid at a given site are contemplated; thus, a codon for the amino acid alanine, a hydrophobic amino acid, can readily be substituted by a codon encoding another hydrophobic residue, such as glycine, or may be substituted with a more hydrophobic residue such as valine, leucine or isoleucine. Similarly, changes which result in a substitution of a negatively charged residue for another, such as aspartic acid for glutamic acid, or one positively charged residue for another, such as lycine for arginine, can also be expected to produced a biologically equivalent product.

The invention also encompasses chimaeric genes, in which the substituted portion of the corn or Arabidopsis AHAS gene is recombined with unaltered portions of the normal AHAS gene, in which the substituted portion of the corn or Arabidopsis AHAS gene is recombined with unaltered portions of the normal AHAS gene from other species. Thus, throughout the specification and claims, wherever the term “herbicide resistant AHAS gene” is used, it is intended to cover each of these alternate embodiments as well as the sequence of SEQ-ID No. 1.

For expression of the mutated AHAS gene conferring herbicide resistance in potato the following promoters can be used:

the tuber specific gbss-promoter from potato described in WO 92/11376, and the patatin promoter described in Rocha-Sosa et al., 1989 EMBO J. 8:23-29;

the light inducible promoter: cytosolic FBPase from potato described in WO 98/18940;

the octopine promoters (U.S. Pat. No. 5,428,147); the triple OCS enhanced vATPase c1 promoter from Beta vulgaris (Plant Mol Biol (1999) 39: 463-475);

Constitutive promoters: for reference see Benfey et al., EMBO J. 8 (1989), 2195-2202; the 35S promoter (Franck et al., Cell 21, (1980), 285-294) or enhanced versions; the 19S promoter, see U.S. Pat. No. 5,352,605 and WO 84/02913;

the RUBISCO small subunit SSU promoter: see U.S. Pat. No. 4,962,028;

plastid specific promoter: see e.g. the RNA polymerase promoter in WO 95/16783 and WO 97/06250 or the clpP-promoter in WO 99/46394;

the AHAS promoter as described in U.S. Pat. No. 6,025,541;

Other promoters for the expression of genes in the leaf, in the callus, in specific tissues as e.g. in the tubers or other parts of the potato plant could also be used.

The invention can especially be carried out by using the AHAS promoter, the AHAS resistance gene S653N as described in U.S. Pat. No. 5,767,366 and the AHAS terminator from Arabidopis thaliana cut from pAC321 as described in example 3.

The Arabidopsis AHAS gene (S653N) used for transformation and selection contains most of the common restriction sites for cloning such as HindIII, BamHI, EcoRI, SstI, BglII and EcoRV. Alteration of the molecular composition of the AHAS gene can be performed to eliminate restriction sites without altering the amino acid sequence of the resulting protein. Special care must be taken not to alter the codon usage profile from the one found in Arabidopsis, which could lead to reduced translation efficiency.

The designed gene can be made synthetically and accommodates suitable restriction sites at 5′ and 3′ ends for cloning.

Comparative studies have to be performed in potato in order to confirm that the synthetically made gene does not differ from the original mutated AHAS gene in conferring herbicide resistance.

For selection of transgenic potato plants chemical compounds inhibiting the AHAS enzyme can be used. Usefull compounds are the imidazoline type herbicides. Especially useful compounds are selected from the group consisting of imazethapyr (Pursuit™), imazamox (Raptor™), imazamethabenz (Assert™), imazapyr (Aresenal™), imazapic (Cadre™) and imazaquinon (Scepter™)

For selection of transgenic plants chemical compounds as described in the review article by Duggleby, R. G. and Pang, S. S. in Journal of Biochemistry and Molecular Biology 33(1), 1-36 (2000) can be used.

The mutated AHAS gene as described in SEQ-ID No. 1 has been established as a selection marker in five different potato varieties, see table 2 and 3, and can thus be used for transformation of potato varieties in general. Two different selection pressures, using 0.3 and 0.5 μM Imazamox, have been tested, see table 2. For selection on 0.3 μM Imazamox the transformation efficiency is ranging from 7 to 76% depending on the variety used. When increasing the selection to 0.5 μM Imazamox the transformation rate is increasing to 93 to 100% respectively. The high transformation efficiency at 0.5 μM Imazamox is independent from the variety used. The fifth variety Seresta has as can be seen in table 3 and 4 a transformation efficiency of 98 to 100% at 0.5 μM Imazamox.

The invention furthermore relates to a plant expression vector comprising SEQ-ID No. 1 wherein the heterologeous DNA sequence encodes a peptide, protein, antisense-, sense-RNA, viral RNA or ribozyme.

The invention furthermore relates to a plant expression vector comprising SEQ-ID No. 1 wherein the heterologeous DNA sequence contains information that causes changes in the carbohydrate concentration and the carbohydrate composition of regenerated potato plants.

The invention furthermore relates to a plant expression vector comprising SEQ-ID No. 1 wherein the heterologeous DNA sequence contains information that causes the increased production of amylose type starches by using gene constructs as described in WO 92/11375, WO 92/14827, WO 96/34968 and WO 97/20040.

The invention furthermore relates to the use of a DNA sequence SEQ-ID No. 1 or a DNA sequence comprising a nucleotide sequence which hybridizes to a complementary strand of the nucleotide SEQ-ID No. 1 or a DNA sequence comprising a nucleotide sequence which is degenerated to the nucleotide sequence SEQ-ID No. 1 or a DNA sequence being a derivative, analogue or fragment of a nucleotide sequence encoding a protein possessing AHA synthase activity and conferring resistance to AHA synthase inhibitors.

The experiment shown in example 12 with the high amylopectin trait establishes the use of the AHAS resistance gene conferring resistance to herbicides inhibiting the AHAS enzyme as a selection marker also for commercially important traits. The transformation efficiency is 98 and 100% respectively. Analysis of the amylopectin/amylose ratio shows that in the transgenic shoots selected the amylopectin content is higher than in non transgenic control shoots. This is proof that by using the invention as described commercially relevant transgenic potato plants can be generated.

The invention now having been generally described will be more readily understood by reference to the following examples, which are included for the purpose of illustration only, and are not intended to limit scope of the present invention.

### EXAMPLE 1

Test for Transgenicity—Detection of Glucuronidase Expression

Microtubers of the regenerated in vitro potato plants were subjected to a qualitative glucuronidase (GUS) enzyme detection by infiltrating cut microtubers for 1 minute in vacuo with the GUS substrate 5-bromo-4-chloro-3-indolyl-β-D-glucuronic acid (X-GlcA) in a 100 mM sodium phosphate buffer, pH 7.0, which contained 10 mM EDTA, 0.1% Triton X100 and 10 mM DTT, and subsequently incubating them for approximately 15 hours at 37° C. Microtubers showed an intense blue coloration, which proves that the reporter gene is expressed in Solanum.

### EXAMPLE 2

Investigation of Selection Pressure

Potato leaf segments from in vitro propagated plants of four different potato varieties Prevalent, Producent, Karnico and Desiree were tested for natural tolerance on different concentrations of the imidazolinone herbicide Imazamox. It is important to choose a selection concentration at which the potato leaf tissue is viable long enough to regenerate shoots but have a high enough concentration to prevent the regeneration of untransformed shoots. Doses 0.1, 0.5, 1, 5, 10, 20, 30 and 50 μM Imazamox were tested.

Fully expended potato leaves are diagonally cut in 2 pieces and precultivated on MC-plates for 2 to 3 days at 23 to 24° C., see table 1.

The leaf tissues are transferred on MS300 medium for additional 2 days and cultivated under modest light at 23 to 24° C. simulating a co-cultivation step. Subsequently the leaf segments are moved to MS400 plates containing 400 mg/l Claforan. Claforan is added in order to suppress growth of Agrobacterium tumfaciens. Therefore the regeneration of shoots on different concentrations of Imazamox need to be monitored in presence of Claforan. Selection is taking place within 4 to 5 days when the explants are moved to MS400 medium supplemented with 400mg/l Claforan and Imazamox at above concentrations.

The explants are transferred to fresh selection medium every fortnight.

TABLE 1

Media used for potato transformation
MC plates
MS300 plates with 1.5-2 ml
liquid MS100 medium and covered with one sterile
filter paper
MS 10
4.4 g/l MS-medium (Murashige and Skoog)
1% (w/v) sucrose
pH 5.8
MS 30
4.4 g/l MS-medium
3% (w/v) sucrose
pH 5.8
MS 100
4.4 mg/l MS-medium
30 g/l sucrose
0.5 mg/l thiamin-HCl
0.5 mg/l pyridoxin-HCl
1 mg/l nicotinacid
0.5 mg/l kinetin
29.8 mg/l ferrous sulfate
hepta hydrate
1 mg/l 2,4-Dichlorophenoxyacetic acid
2 g/l caseinhydrolysate
pH 5.2
MS300
X MS-medium
2 mg/l NAA (naphtyl acetic acid)
1 mg/l BAP (6-benzyl amino pyridine)
3% (w/v) sucrose
pH 5.2
MS400
4.4 g/l MS-medium
2 mg/l zeatine
0.01 mg/l NAA (naphtyl acetic acid)
0.1 mg/l GA3 (giberellic acid)
10% (w/v) sucrose
400 mg/l claforan
Imazamox or kanamycin
pH 5.8
Microtuber medium
4.4 g/l MS-medium
2.5 mg/l kinetin
0.5 mg/l ABA (abscisic acid)
8% sucrose
200 mg/l claforan

### EXAMPLE 3

Construction of Binary Vectors pAHASGUS, pAP1 and pAP2

For all constructs the same AHAS mutant gene with mutation S653N originating from Arabidopis thaliana was used (Sathasivan, K. et al., 1991).

pAHASGUS

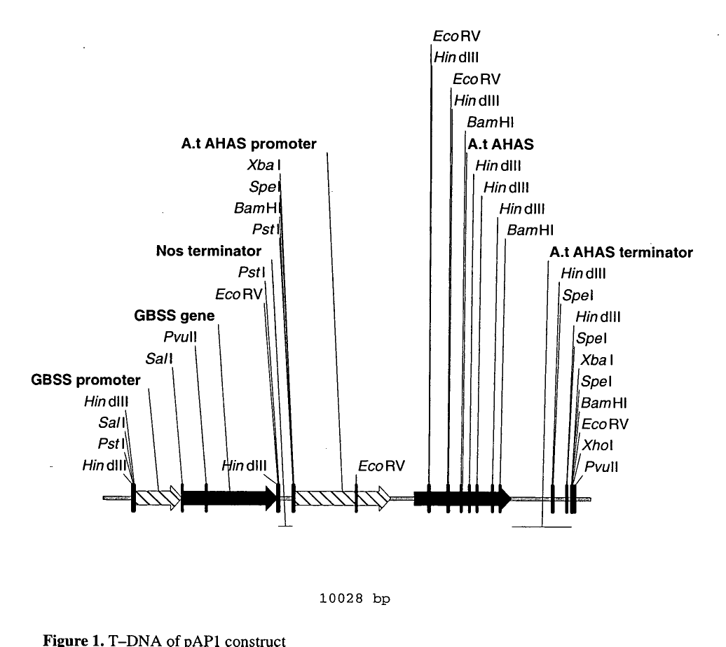

AHAS gene with mutation S653N as described in SEQ ID No. 1, originating from Arabidopis thaliana, was used together with the nos promoter (Herrera, L. et al., 1983) and the OCS terminator (Wesley S. V. et al., 2001) in the binary vector pGPTVkan (Becker, D. et al., 1992). The pBIN19 based pGPTVkan was digested with ApaI and BamHI discarding the nptII gene. The remaining 12600 bp contained from right border the nos terminator, the uidA gene (also known as GUS), the cloning cassette, the nos promoter and the pAg7 terminator. The AHAS gene with mutation S653N and the OCS terminator was ligated to the digested pGPTVkan and named pABA1, see FIG. 1.

For expression of the GUS gene a tuber specific gbss promoter 988 bp (WO 92/11376) was cloned at the SmaI site of pABA1.

The resulting pAHASGUS construct was used for Agrobacterium tumfaciens transformation of potato, where regenerated plantlets were analysed for GUS expression to determine the transformation efficiency.

pAP1

AHAS gene with mutation S653N, driven by the Arabidopis thaliana AHAS promoter as described in U.S. Pat. No. 5,750,866, was used as a selection gene for co-transformation with a granule-bound starch synthase (gbss) antisense gene (EP-A 0 563 189).

A 293 bp nos terminator was cut out from pHAxwO and ligated to pbluescript with EcoRI. The 2952 bp gbss promotor and gbss gene in antisense direction (EP-A 0 563 189) was cut from pHAxwO and ligated in front of the nos terminator with HindIII resulting in pMJ2. The gbss complex was cut out from pMJ2 with XbaI-XhoI and ligated to pSUN1 (WO 02/00900) cut with XbaI-SalI resulting in the construct named pMJ3.

pAC321 was cut with XbaI which yielded a fragment of 5717 bp containing AHAS promoter, AHAS gene and AHAS terminator all originating from Arabidopis thaliana as described in SEQ-ID No. 1.

SEQ-ID No.1 contains the following elements:
1-2483 At AHAS promoter
2484-4496 At AHAS gene
4497-5717 At AHAS terminator

The pAC321 fragment was ligated to pMJ3 opened with XbaI.

The resulting pAP1 (FIG. 1) construct was used for Agrobacterium tumefaciens transformation of potato, which were analysed for high amylopectin starch quality.

pAP2

AHAS gene with mutation S653N, driven by the nos promoter (U.S. Pat. No. 6,174,724), was used as a selection marker for co-transformation with a gbss antisense gene (WO 92/11376).

A nos promotor of 600 bp was cut out from pGPTVKan (Becker et al., 1992) with HindIII, blunted and cut with BglII. The fragment was ligated to pBluescript (Stratagene) cut with SpeI, blunted and cut with BamHI. The construct was named pPnos2.

A 2986 bp fragment of pACGH101 containing the AHAS gene with mutation S653N as described in SEQ ID No. 1 and OCS terminator was cut out with PstI and PvuII. The AHAS gene originates from Arabidopis thaliana see pAP1. The pACGH101 fragment was ligated to pPnos2 cut with PstI and EcoRV and named pPnoas.

The 3406 bp AHAS complex was cut out from pPnoas with XbaI-PvuII and ligated to pMJ3 (see pAP1) cut open with XbaI.

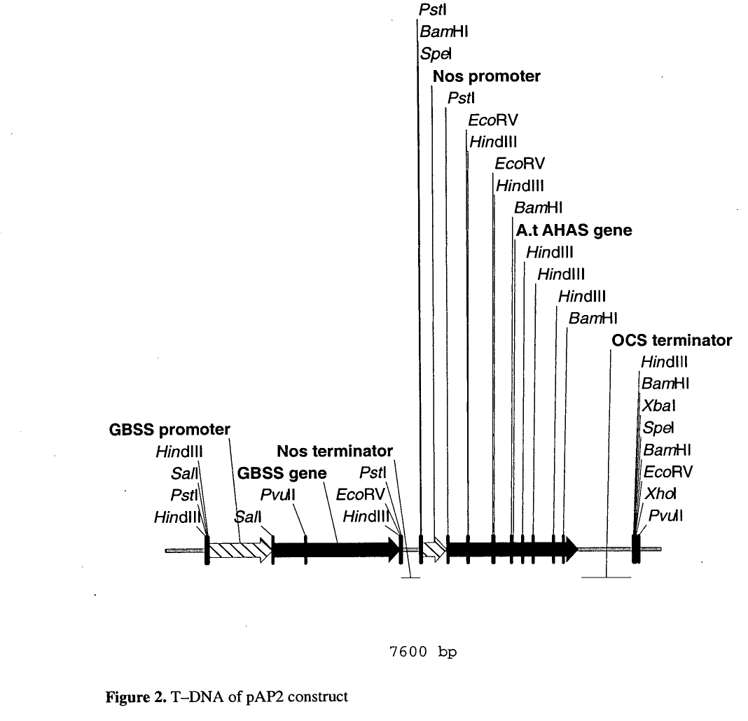

The resulting pAP2 (FIG. 2) construct was used for Agrobacterium tumefaciens transformation of potato, which were analysed for high amylopectin starch quality.

### EXAMPLE 4

Transformation Method

Fully expanded leaves from in vitro propagated potato plants are diagonally cut in 2 pieces and precultivated on MC-plates for 2 to 3 days at 23 to 24° C.

Agrobacterium tumfaciens containing pAHASGUS is grown in YEB medium containing 25 μg/ml kanamycin and 100 μg rifampicin and grown over night on constant shaking (200 rpm) at 28° C.

The Agrobacterium culture is prepared for infection by dilution 1:20 with MS10 medium. The leaf explants are infected for 8-10 min in the bacterial solution and afterwards drained on filter paper for 5 to 20 seconds. The leaf segments are placed on the MS300 plates for 2 days co-cultivation under modest light at 23 to 24° C. At the end of co-cultivation the leaf segments are moved to M400 plates containing 400 mg/l Claforan to suppress bacterial growth. After 4 to 5 days the explants are moved to selection medium MS400 supplemented with 400 mg/l Claforan and Imazamox at the previously detected concentrations of 0.3 or 0.5 μM.

Leaf segments are transferred to fresh MS 400 selection medium every fortnight. The regenerated putative transgenic shoots are collected and cultivated on MS30 plates with 200 mg/l Claforan aiming at shoot elongation.

When the shoots are 3 to 5 cm long, 1 to 2 cm are cut off and grown on microtuber medium in the dark at 25° C. After 2 to 5 weeks microtubers are produced. Putative transgenic plants are analysed for GUS expression in microtubers to determine the transformation efficiency.

### EXAMPLE 5

GUS Expression Analysis for Determination of the Transformation Efficiency

A thin slice from each microtuber was placed in a microtiter plate. The slices were incubated with 1 mM X-gluc in 50 mM NaH₂PO₄ pH 7.2 at 37° C. for approximately 1 hour. A very distinct blue colour appeared on tuber slices if a successful integration and expression of the GUS gene was achieved.

### EXAMPLE 6

Transformation Efficiency Determination of pAP1 and pAP2 by PCR Analysis

Transgenic shoots were distinguished from non-transgenic shoots—for example escapes—by using PCR. DNA was extracted according to DNeasy 96 Plant protocol (Qiagen, Germany). In a 96 well microtiter plate, 10 to 15 mg leaf tissue was added to each well together with a 5 mm steel ball each well then representing one individual shoot. The plates were frozen in N₂(1) before homogenisation. The homogenisation was done at 30 Hz in a Mixermill 300 for 1 min. The DNA was at the end of the extraction protocol eluted in 75 ml H₂O.

Specific primers were used to amplify a fragment of the mutant S653N AHAS gene. Successful integration of the mutant AHAS gene results in a fragment of 509 bp upon PCR amplification using the specific primers.

Forward primer: AHAS1_frw: AACAACAACATCTTCTTCGATC
Reverse primer: AHAS1_rev: TAACGAGATTTGTAGCTCCG

The PCR reactions were done with the extracted DNA setup and run as follows:

Reaction:

| | | |
|---|---|---|
| 10x PCR Mix | 2.0 | μl |
| Primer frw (25 μM) | 0.4 | μl |
| Primer rev (25 μM) | 0.4 | μl |
| dNTPs (10 mM) | 0.4 | μl |
| RedTAQ (Sigma) | 1.0 | μl |
| Templat (˜20 ng/μl) | 4.0 | μl |
| H₂O | 11.8 | μl |

PCR Program:

| | |
|---|---|
| 94° C. | 30 s |
| 59° C. | 30 s × 29 cycles |
| 72° C. | 30 s |
| 72° C. | 7 min |
| 8° C. | Hold |

Negative and positive control was included in all runs. The reactions were analysed on 1.5% agarose gels.

### EXAMPLE 7

Starch Quality Analysis of Amylopectin/Amylose Ratio

The screening for an altered amylopectin/amylose ratio was performed on transgenic lines selected for high quality integration of the intended genetic insert. The screening was done by staining with iodine (Lugol's solution: (6.7 g/l KI+3.3 g/l I₂) and (glycerol); ratio 1:1). Iodine stains starch containing amylose blue and starch exclusively containing amylopectin red-brown. A microtuber was crushed and a few drops of Lugol's solution were added. The starch amylopectin quality was analysed under the microscope according to the staining colour of the starch.

### EXAMPLE 8

Leaf tissues were tested for the use of the AHAS resistance gene contained in SEQ ID No. 1 and for the selection of transgenic potato plant cells as described above. Four different potato cultivars Prevalent, Producent, Karnico and Desiree were cultivated on different concentrations of Imazamox, an imidazolinones type herbicides. It is essential to use the proper concentration for selection to yield an acceptable number of shoots per explant without high escape rate. The lethality of various Imazamox concentrations on regeneration was evaluated.

Lethal dose for regeneration was detected within 0.1 to 1 μM ranges of concentrations. Slight variation in lethality was detected between varieties. No regeneration occurs when 0.5 μM Imazamox was included in the medium. The concentrations 0.3 μM and 0.5 μM are selected for transformation experiments providing a lethality of 80 and 100% in regeneration.

### EXAMPLE 9

Transformation of four different potato varieties (Prevalent, Producent, Kuras and Desiree) was performed with pAHASGUS as described above. For selection a concentration of 0.3 or 0.5 μM Imazamox was used. Each shoot was analysed according to GUS activity and the number of GUS positive shoots in total is listed in table 2. A distinct blue colour appears on the microtuber slices if a successful integration of the GUS gene was achieved. From each explant 3 to 5 independent shoots were regenerated depending on the variety used. In all experiments the efficiency of positive shoots per explants is stated and the figures in table 2 have to be multiplied 3 to 5 times, yielding a transformation efficiency of up to 500%. Compared with selection systems used previously in potato plants the big advantage of the invention described is that together with a very high efficiency the number of shoots per explant is very high. For all four varieties transformed an extremely high transformation efficiency of up to 93% to 100% of all shoots regenerated was achieved at an Imazamox concentration of 0.5 μM. The experiment using 0.3 μM of Imazamox for selection shows how important the Imazamox concentration is for an efficient transformation rate. This can be seen from the results for variety Kuras in which a transformation efficiency of 7% at 0.3 μM Imazamox is increased to 93% at the 0.5 μM level.

TABLE 2
Transformation efficiency of four different varieties with
0.3 and 0.5 μM Imazamox used as the selective agent.

| | | Selection | Selection | | | |
|---|---|---|---|---|---|---|
| | | Imazamox | Imazamox | Number | Number | (%) |
| Variety | Construct | (μM) | (mg/l) | analysed | of positive | positive |
| Desiree | pAHASGUS | 0.3 μM | 0.0915 mg/l | 19 | 12 | 63 |
| Prevalent | pAHASGUS | 0.3 μM | 0.0915 mg/l | 26 | 16 | 62 |
| Producent | pAHASGUS | 0.3 μM | 0.0915 mg/l | 25 | 19 | 76 |
| Kuras | pAHASGUS | 0.3 μM | 0.0915 mg/l | 58 | 4 | 7 |
| Prevalent | pAHASGUS | 0.5 μM | 0.1525 mg/l | 23 | 23 | 100 |
| Producent | pAHASGUS | 0.5 μM | 0.1525 mg/l | 40 | 38 | 95 |
| Kuras | pAHASGUS | 0.5 μM | 0.1525 mg/l | 33 | 32 | 97 |
| Desiree | pAHASGUS | 0.5 μM | 0.1525 mg/l | 29 | 27 | 93 |

### EXAMPLE 10

In a similar experiment compared to example 9 the nptII gene was used as selection marker being controlled by the same promoter nos as in pAHASGUS. The shoots are selected on 50 mg/l kanamycin, which is a standard kanamycin concentration used for potato transformation as described in Ooms, G. et al., 1987 and Tavazza, R. et al., 1988. The transformation frequency was much lower compared to when using a mutated AHAS gene e.g. the S653N gene as selection marker. The highest transformation efficiency based on analysis of the same GUS gene driven by the same GBSS promoter resulted in 74% transformed shoots regenerated.

### EXAMPLE 11

The successful use of a mutated AHAS gene as a selection marker has been demonstrated using the promoter from the wild-type Arabidopis thaliana AHAS gene (pAP1 construct) as well as a recombinant nos promoter (pAP2 construct) in the potato variety Seresta, see table 3. This shows that any promoter with sufficient expression can be used in connection with the mutated AHAS gene and thus the invention could be used with different promoters as regulatory elements for the mutated AHAS gene successfully yielding selection of transgenic shoots on selective medium. The AHAS gene can be driven by different promoters such as e.g. the AHAS-, nos-, 35S-, Rubisco- or vATPase-promoter.

TABLE 3
Transformation efficiency of the mutated AHAS gene driven
by two different promoters: Arabidopsis thaliana (A.t.)
AHAS promoter and the nos promoter.

| | | | Number | Number | |
|---|---|---|---|---|---|
| | | | shoots | shoots | % |
| Variety | Construct | Promoter | analysed | positive | positive |
| Seresta | pAP1 | A.t AHAS | 50 | 49 | 98 |
| Seresta | pAP2 | nos | 120 | 117 | 98 |

### EXAMPLE 12

The mutated AHAS gene conferring resistance to Imazamox was used as a selection marker for the transformation of a commercially important trait to Solanum tuberosum (variety Seresta) resulting in potato lines producing amylopectine type starch. The starch component amylose is synthesized by granule-bound starch synthase (GBSS). Inhibition of the gene coding for GBSS directs starch production completely to amylopectin. An antisense gene fragment inhibiting the expression of gbss as described in EP-A 0 563 189 was co-transformed with the mutated AHAS gene conferring resistance to Imazamox.

The gbss promoter was used to drive the gbss antisense gene fragment. The Nos promoter or the AHAS promoter was used to drive the AHAS selection gene and resulted in a total transformation efficiency per shoots analysed of 98 to 100%, see table 4.

For restriction map of the T-DNA of the pAP1 and pAP2 constructs used, see FIG. 1 and 2. There were 3 to 5 independent shoots produced per explant yielding a transformation efficiency of 280 to 480% or up to 500% per explant. There were no differences in transformation efficiency between the different promoters and Solanum tuberosum varieties used.

TABLE 4
pAP1 and pAP2 transgenic lines analysed for transformation
efficiency and high amylopectin quality.

| | | | | | | high |
|---|---|---|---|---|---|---|
| | | | Number | Number | % | amylo- |
| | | | shoots | shoots | posi- | pectin |
| Experiment | Variety | Construct | analysed | positive | tive | content |
| 1 | Seresta | pAP2 | 120 | 117 | 98 | + |
| 2 | Seresta | pAP1 | 50 | 49 | 98 | + |
| 3 | Seresta | pAP2 | 30 | 30 | 100 | + |
| 4 | Seresta | pAP2 | 3 | 3 | 100 | + |

Starch quality analysis was done with microtubers of the positive shoots with high quality integration of the intended genetic insert. The starch was mixed with iodine and the shoots exclusively containing amylopectin stains red-brown compared to the starch containing amylose that stains blue.

### REFERENCES

Hawkes, T. R., in Prospects for Amino Acid Biosynthesis Inhibitors in crop protection and Pharmaceutical Chemistry, pp 131-138, Brittish Crop Protection Council Monograph No 42, Surrey, U.K. (1989)
Herrera-Estrella, L., Depicker, a., Van Montagu, M., Schell, J., Expression of chimaeric genes transferred into plant cells using Ti-plasmid-derived vector, Nature 303:209-213 (1983)
Becker, D., Kemper, E., Schell, J., Masterson, R., New plant binary vectors with selectable markers located proximately to the left T-DNA border, Plant Molecular Biology 20:1195-1197, (1992)
Jefferson, R. A., Kavanagh, T. A., Bevan, M. W., GUS fusions: b-glucuronidase as a sensitive and versatile gene fusion marker in higher plants, EMBO Journal 6: 3901-3907 (1987)
Joersbo, M., Donaldson, I., Kreiberg, J., Guldager Petersen, S., Brunstedt, J., and Okkels, F. T., Analysis of mannose selection used for transformation of sugar beet, Molecular Breeding 4:111-117 (1998)
Libiakova, G., Jorgensen, B., Palmgren, G., Ulskov, P., Johansen, E., Efficacy of an intron containing kanamycin resistance gene as a selectable marker in plant transformation, Plant Cell Rep 20:610-615(2001)
Ooms, G., Burell, M. M., Karp, A., Bevan, M., Hille, J., Genetic transformation in two potato cultivars with T-DNA from disarmed Agrobacterium, Theoretical and Applied Genetics (1987) 73:744-750,
Ray, T. B., Plant Physiology (1984), 75, 827-831
Sathasivan, K., Haughn, G. W., Murai, N., Plant Physiology 97(1991), 1044-1050
Shaner, D. L., Andersson, P. C., Stidham, M. A., Plant Physiology 76 (1984), 545-546
Subrimanian, M. V., Gerwick, B. C., in Biocatalysis in Agricultural Biotechnology, pp 277-288, ACS Symposium Series No389, American Chemical Society, Washington D.C. (1989)
Tavazza, R., Tavazza, M., Ordas, R. J., Ancora, G., Benvenuto, E., Genetic transformation of potato (Solanum tuberosum): An efficient method to obtain transgenic plants, Plant Science, 59 (1988), 175-181
Umbarger, H. E., in Synthesis of Amino Acids and Proteins, (1975) pp 1-56, MTP International Review of Science, Butterworth, London.
Wesley S. V., Helliwell C. A., Smith N. A., Wang M. B., Rouse D. T., Liu Q., Gooding P. S., Singh S. P., Abbott D., Stoutjesdijk P. A., Robinson S. P., Gleave A. P., Green A. G., Waterhouse P. M., Construct design for efficient, effective and high-throughput gene silencing in plants, Plant J September 2001;27(6):581-90
Haldrup A, Petersen, G P, Okkels F T (1998) The xylose isomerase gene from Thermoanaerobacterium thermosulfurogenes allows effective selection of transgenic plant cells using D-xylose as the selection agent. Plant Molecular Biology 37:287-296

1. A method for transformation of potato plants by transforming potato plant cells with an expression vector comprising
a) regulatory sequences of a promoter active in plants;
b) operably linked thereto a DNA sequence encoding a protein with the biological activity of an AHA synthase resistant to inhibitors of potato plant wildtype AHA synthase; and
c) operably linked thereto regulatory sequences which serve as transcription termination and/or polyadenylation signals in plants,
selecting for AHA synthase inhibitor resistant cells and regenerating them to transgenic plants.

2. The method for transformation according to claim 1, wherein the expression vector comprises the DNA sequence of SEQ ID NO: 1.

3. The method for transformation according to claim 1, wherein the DNA sequence encoding a protein with the biological activity of an AHA synthase resistant to inhibitors of potato plant wildtype AHA synthase is selected from the group consisting of
a) a DNA sequence comprising the nucleotide sequence of SEQ ID NO: 1;
b) a DNA sequence comprising a nucleotide sequence which hybridizes to a complementary strand of the nucleotide sequence of a);
c) a DNA sequence comprising a nucleotide sequence which is degenerate to the nucleotide sequence of a); and
d) a DNA sequence being a derivative, analogue or fragment of a nucleotide sequence of a), b) or c) and encoding a protein possessing AHA synthase activity and conferring resistance to AHA synthase inhibitors.

4. The method for transformation according to claim 1, wherein a AHA synthase promoter from Arabidopis thaliana or nos promoter is used.

5. The method for transformation according to claim 1, wherein a AHA synthase terminator from Arabidopis thaliana or a OCS terminator is used.

6. The method for transformation according to claim 1, wherein for selection a imidazolinone type herbicide is used.

7. The method for transformation according to claim 6, wherein for selection (RS)-2-(4-isopropyl-4-methyl-5-oxo-2-imidazolin-2-yl)-5-methoxymethylnicotinic acid is used.

8. The plant expression vector according to claim 1 additionally comprising a heterologous DNA sequence.

9. The plant expression vector according to claim 8, wherein the heterologous DNA sequence encodes a peptide, protein, antisense-, sense-RNA, viral RNA or ribozyme.

10. The plant expression vector according to claim 9, wherein the heterologous DNA sequence contains information that causes changes in the carbohydrate concentration and the carbohydrate composition of regenerated potato plants.

11. The plant expression vector according to claim 10, wherein the heterologous DNA sequence contains information that causes increased production of amylopectin type starches.

12. The plant expression vector according to claim 10, wherein the heterologous DNA sequence contains information that causes increased production of amylose type starches.

13. A transgenic potato plant cell produced by the method for transformation according to claim 1 and containing the plant expression vector according to claim 1 additionally comprising a heterologous DNA sequence.

14. A transgenic potato plant produced by the method of transformation according to claim 1, wherein the regenerated plant exhibits an elevated resistance to imidazolinone type herbicides.

15. A harvest product of the transgenic potato plant according to claim 14 comprising the DNA sequence of SEQ ID NO: 1.

16. The harvest product according to claim 15 wherein the harvest product is a tuber.

17. Propagation material of transgenic potato plants comprising the DNA sequence of SEQ ID NO: 1.

18. Potato plant cells, potato tissue cultures, potato plants and/or potato plant breeding comprising the DNA sequence of SEQ ID NO: 1.

19. A harvest product of the transgenic potato plant according to claim 14 comprising a DNA sequence encoding a protein with the biological activity of an AHA synthase resistant to inhibitors of potato plant wildtype AHA synthase selected from the group consisting of
a) a DNA sequence comprising the nucleotide sequence of SEQ ID NO: 1;
b) a DNA sequence comprising a nucleotide sequence which hybridizes to a complementary strand of the nucleotide sequence of a);
c) a DNA sequence comprising a nucleotide sequence which is degenerate to the nucleotide sequence of a); and
d) a DNA sequence being a derivative, analogue or fragment of a nucleotide sequence of a), b) or c) and encoding a protein possessing AHA synthase activity and conferring resistance to AHA synthase inhibitors.
